(19) United States
(12) Patent Application Publication
(10) Pub. No.: US 2018/0311428 A1
(43) Pub. Date: Nov. 1, 2018

(54) BLOOD TREATMENT DEVICE AND METHOD OF PREPARING A PRESCRIPTION

(71) Applicant: FRESENIUS MEDICAL CARE DEUTSCHLAND GMBH, Bad Homburg (DE)

(72) Inventor: Erik GRIESSMANN, Schweinfurt (DE)

(73) Assignee: FRESENIUS MEDICAL CARE DEUTSCHLAND GMBH, Bad Homburg (DE)

(21) Appl. No.: 15/769,543

(22) PCT Filed: Oct. 19, 2016
(86) PCT No.: PCT/EP2016/001732

§ 371 Date: Apr. 19, 2018

(30) Foreign Application Priority Data
Oct. 20, 2015 (DE) 10 2015 013 627.5

(51) Int. Cl. A61M 1/16; G16H 20/40; G16H 40/63; A61M 1/28

(52) U.S. Cl. CPC A61M 1/1603; G16H 20/40; A61M 2205/505; A61M 1/28; G16H 40/63

(57) ABSTRACT

The present invention relates to a blood treatment device having at least one touch screen for inputting data by the user of the blood treatment device and having at least one memory for storing a prescription, wherein the blood treatment device has at least one processor which is connected both to the touch screen and to the memory and which is configured to determine a prescription based on a progression drawn on the touch screen by the user, wherein the processor is furthermore configured only to determine this prescription based on the progression drawn by the user and without using values of the prescription stored in the memory in advance.

The present invention relates to a blood treatment device having at least one touch screen for inputting data by the user of the blood treatment device and having at least one memory for storing a prescription.

It is known from the prior art that the user of a blood treatment device prepares a prescription via a touch screen according to which the blood treatment is then carried out. Such a procedure is known, for example, from EP 597 817 A2 and from EP 0 668 793 B1. WO 2013/091814 A2 discloses a blood treatment device in which the prescription process is prepared such that the user selects graphic symbols which are stored in a selection region and can then be dragged into a so-called storage region. A prescription progression is prepared on the basis of the symbols located in the storage region which is used as the basis for the treatment.

It is the underlying object of the present invention to further develop a blood treatment device, in particular a blood treatment device having an extracorporeal blood circuit, and preferably a peritoneal dialysis machine or also a hemodialysis machine, such that the preparation of a prescription is simplified.

This object is achieved by a blood treatment device having the features of the claim. Provision is made in this respect that the blood treatment device has at least one processor which is data-linked both to the touch screen and to the memory and which is configured to determine a prescription using a progression drawn on the touch screen by the user, wherein the processor is furthermore configured only to determine this prescription using the drawn progression. No use is made in the preparation of the prescription of any data of the prescription stored prior to the prescription carried out by the user such as a prescription model or of concentrations values relating to a dialysis solution, etc. No data relating to the prescription are preferably present in the memory before the preparation of the prescription by the user.

The present invention is thus based on the idea of preparing the prescription or the prescription progression only be drawing directly on the touch screen, without a prescription model or also other data relating to the prescription, such as concentrations of the administered dialysis solutions, etc., having to be stored in the device beforehand, i.e. before this preparation of the prescription.

The prescription, such as the temporal progression of e.g. the volume of the peritoneal dialysis solution present in the patient is simply prepared in that the user e.g. draws the volume of this solution over time on the touch screen. This prescription is then used as the basis for the treatment.

The term “prescription” is understood within the framework of the invention as the dependence of at least one parameter relating to the blood treatment on at least one other parameter, preferably on the time.

It is neither necessary to store a specific prescription model in the memory in advance, nor is it necessary to input specific data which relate to the prescription in the memory in advance. It is sufficient in accordance with the invention only to determine the prescription using the progression on the touch screen drawn by the user, which does not preclude that changes to the prescription are possible after this determination of the prescription.

A corresponding simplification thereby results with respect to known embodiments of blood treatment devices since the input of values such as concentrations of solutions, of prescription models, etc. which takes place in advance can be dispensed with and a complete new prescription can be prepared in this manner.

The progression can e.g. be the temporal progression of one or more treatment parameters, with provision preferably being made that these treatment parameters are the volume of the dialysis solution present in the patient as part of a peritoneal dialysis treatment, one or more concentrations such as the glucose concentration or the calcium concentration, the ultrafiltration rate or also the clearance.

The treatment parameter(s) can, for example, also be the filling volume and/or the drain volume and/or the dwell time.

This list is of an exemplary nature and is not exclusive. Instead of a temporal progression any other desired dependence of a parameter on another parameter is also conceivable which can be input on the touch screen by the user.

It is pointed out at this point that the feature of the progression drawn on the touch screen by the user comprises both the case that the user draws this progression with his finger and the case that the progression is performed by a drawing tool such as a touch pen which is held by the user.

Provision is made in a further embodiment of the invention that the processor is configured to carry out a vectorization of the drawn progression, i.e. a straightening of the progression. Linear sections are then formed from the drawn progression which are connected to one another at corresponding connection points so that a continuous progression results which is formed by linear sections.

Provision is made in a further embodiment of the invention that the processor is configured to determine a change of the prescription based on one or more changes to the progression made on the touch screen by the user. The user can, for example, change the length of the treatment, the point in time of individual treatment steps, their number, the administered volume of the dialysis solution, the type of treatment, etc. by a corresponding touching of the touch screen. The processor processes this change performed on the touch screen and stores it in the memory.

The device control accesses the memory and carries out the treatment in accordance with the prescription stored there.

The change made by the user is displayed on the touch screen.

The processor can furthermore be configured to generate an output of one or more parameter values relating to the touched section based on a touching of a specific section of the progression on the touch screen by the user or to generate an output which allows the user to add or to cancel treatment steps, in particular treatment cycles.

It can thus be possible, for example, that the user records the volume of the administered dialysis solution over time and which solutions it is, or what the composition of the solution is, is displayed by tapping a section of this progression.

Provision is preferably made in this respect that the user can change these displayed parameter values in that, for example, a selection menu is output which allows the user e.g. to select a different solution or a different solution composition from the selection. Instead of the generation of a selection menu, it is naturally also possible that such a menu is not generated and that the user himself inputs new values for the solution or different parameter values into an input field.

It is also conceivable that the selection menu is designed such that the user can choose from a plurality of solutions from which a mixed solution is prepared. It is thus possible to be able to prepare mixed solutions of different compositions and/or with different concentrations.

The processor can be configured such that it does not allow any changes to the prescription of the already performed part of the treatment.

It is only possible in this case to carry out a change of the prescription such as a change of the still remaining cycles of a peritoneal dialysis treatment, etc. for that part of the treatment which is not yet finished. The already performed part of the prescription can have a different visual design than the still upcoming part of the treatment, for example by a shading or by a color of the screen background or of the progression.

The present invention furthermore relates to a method of preparing a prescription at a blood treatment device, wherein the blood treatment device has a touch screen for inputting data by the user of the blood treatment device as well as at least one memory for storing the prescription. Provision is made in accordance with the invention that the prescription is only prepared in that the user draws the progression of the prescription on the touch screen and that the prescription is thus prepared without values of the prescription being input by the user or being called from the memory in advance in addition to the drawn progression.

As already stated above, it is sufficient for the preparation of the prescription that the user draws the corresponding progression. It is neither necessary to input additional values nor is it necessary to store a prescription model or individual values of the prescription prior to the preparation in accordance with the invention of the prescription.

The method is preferably characterized in that the progression is the temporal progression of one or more treatment parameters, with provision preferably being made that these treatment parameters are the volume of the dialysis solution present in the patient as part of a peritoneal dialysis treatment, one or more concentrations such as the glucose concentration or the calcium concentration, the ultrafiltration rate or also the clearance.

The treatment parameter(s) can, for example, also be the filling volume and/or the drain volume and/or the dwell time.

This list is of an exemplary nature and is not exclusive. Instead of a temporal progression any other desired dependence of a parameter on another parameter is also conceivable which can be input on the touch screen by the user.

A vectorization of the drawn progression is preferably carried out by the device.

The progression or the prescription carried out on the touch screen by the user can preferably be subsequently changed. This change can, for example, relate to the duration, the point in time of individual treatment steps, their number, the administered volume of the dialysis solution or also the type of treatment. It is thus possible, for example, to switch over from conventional peritoneal dialysis to a tidal treatment in that the user shifts one or more points of the prescription accordingly.

As furthermore stated above, it is possible that specific parameter values of the total treatment progression or of the prescription or of individual sections thereof are output to the user. The user has the possibility of changing them in that, for example, a different dialysis solution is selected.

Provision is furthermore made that no changes to the prescription of the already carried out part of the treatment are possible.

Provision can furthermore be made that a button or another selection region, optionally from a menu, is present on the touch screen which can be actuated by the user and whose actuation tells the device that a disconnection break is present in which the patient is not connected to the device. Subsequently to the disconnection break, the patient can again connect himself to the device and continue the treatment.

Provision can be made in a further embodiment of the invention that an information button or another selection region, optionally from a menu, is present on the touch screen which can be actuated by the user and on whose actuation information is output. This information can, for example, be the prompt to the user to take medication, the information relating to the peritoneal function, a free text or also one or more other action instructions to the user. This button or this selection region can be configured such that it can be moved to the desired position on the touch screen by means of drag and drop, for example.

Further details and advantages of the invention will be explained in more detail with reference to an embodiment shown in the drawing.

There are shown:

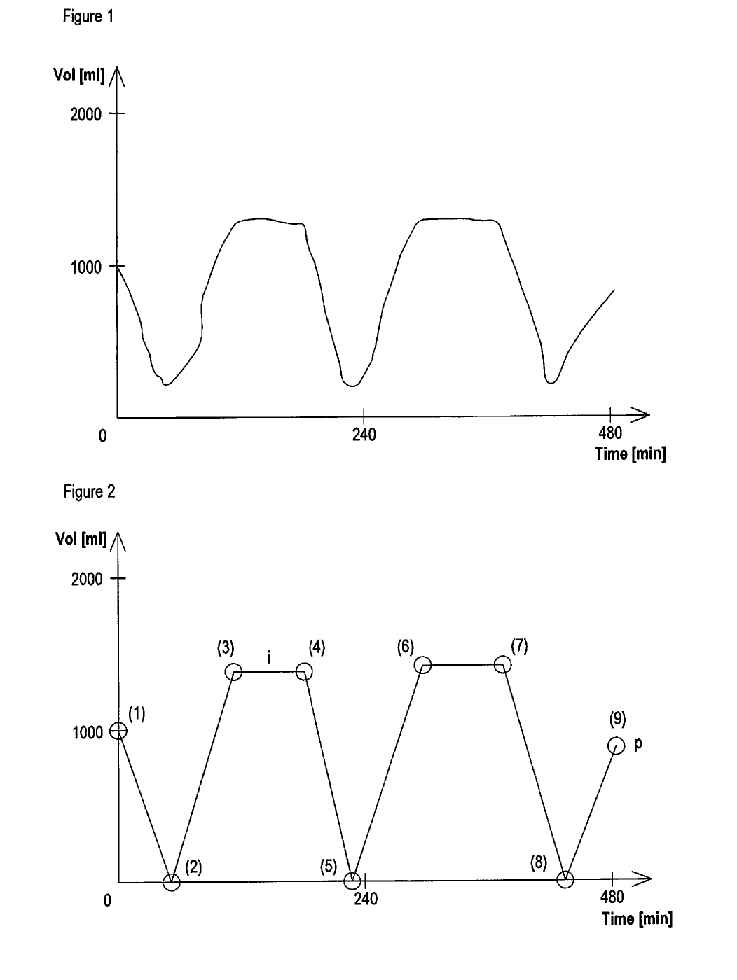

FIG. 1: the temporal progression of the volume of the dialysis solution which is input by the user and which is administered to and removed from the patient as part of a peritoneal dialysis treatment;

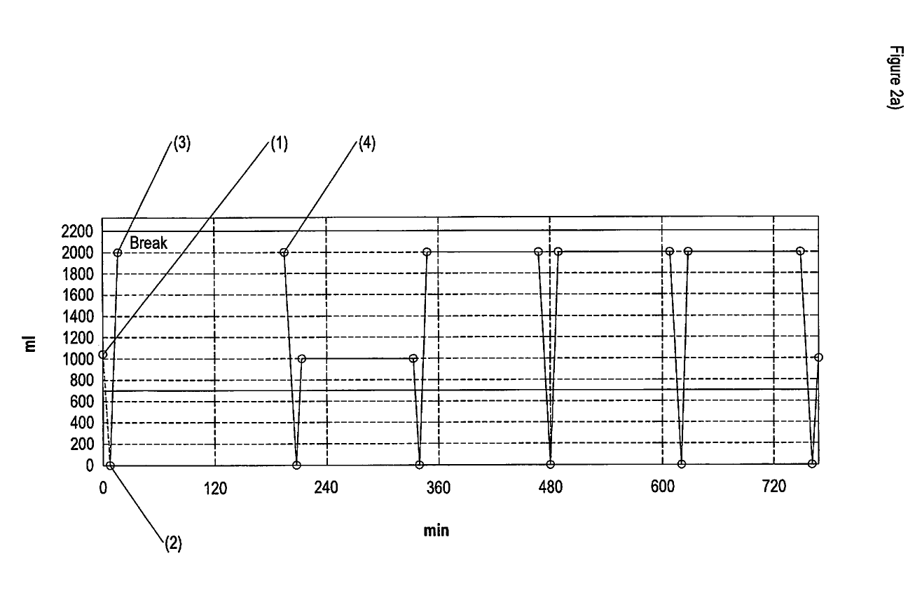

FIG. 2: a view in accordance with FIG. 1 after the vectorization;

FIG. 2a: the temporal progression of the volume of the dialysis solution which is input by the user and which is administered to and removed from the patient as part of a peritoneal dialysis treatment with a disconnection break;

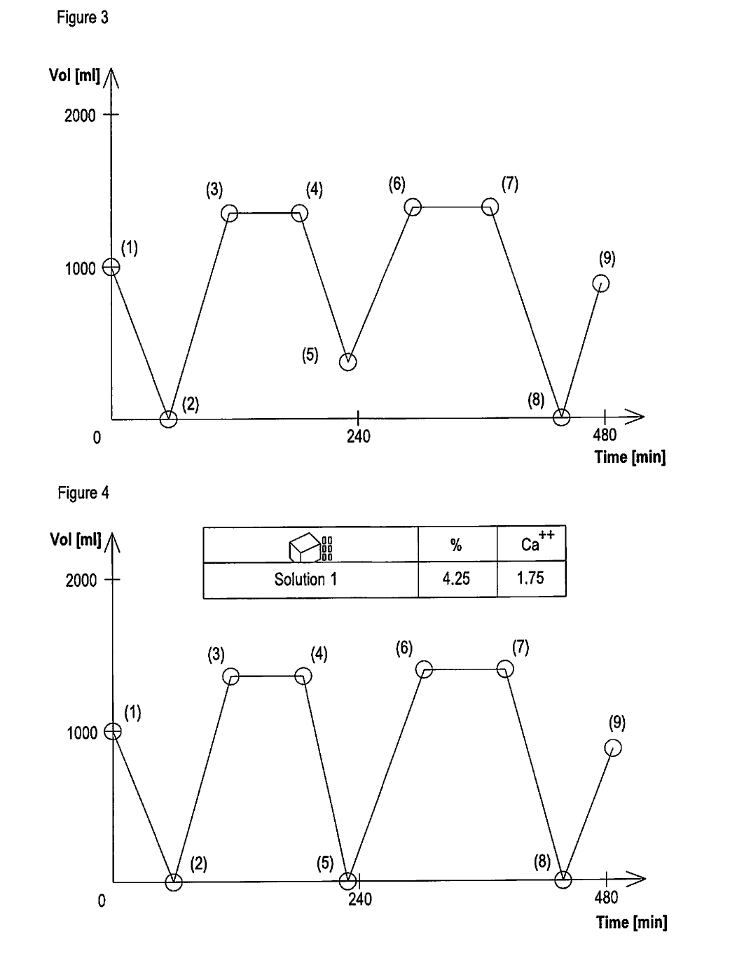

FIG. 3: a view in accordance with FIG. 2 after a change to the treatment type having a tidal cycle;

FIG. 4: a view in accordance with FIG. 2 with an emphasized section of the prescription and a display of the administered solution;

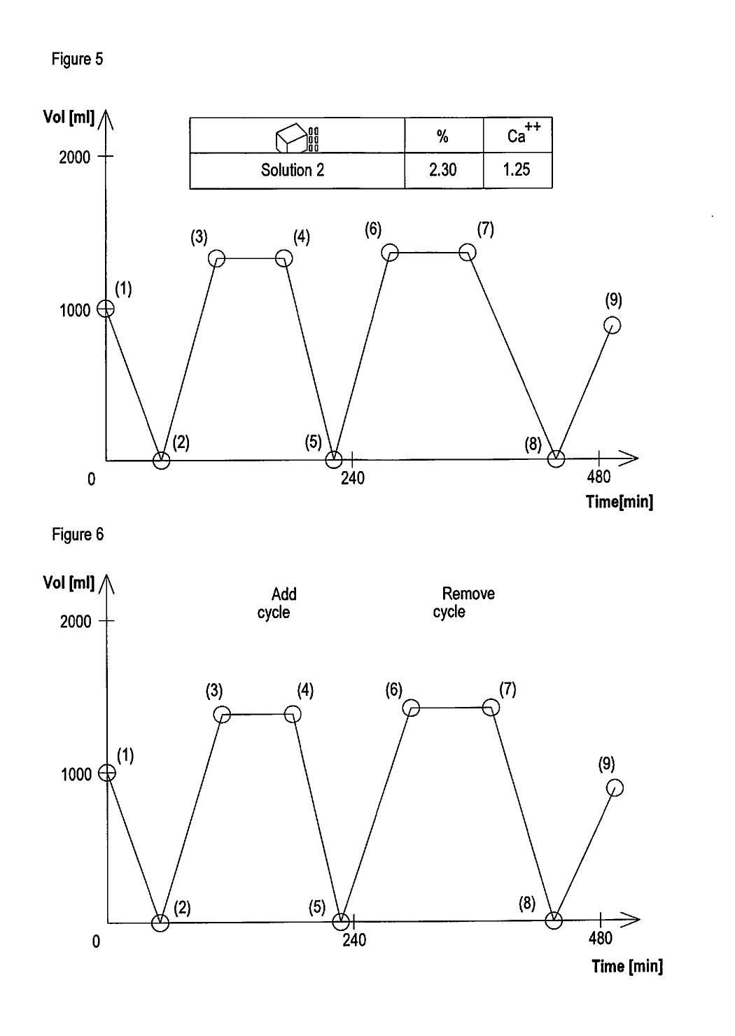

FIG. 5: a view in accordance with FIG. 4 after a change to the dialysis solution by the user;

FIG. 6: a view in accordance with FIG. 2 with the possibility of adding or removing individual treatment cycles; and

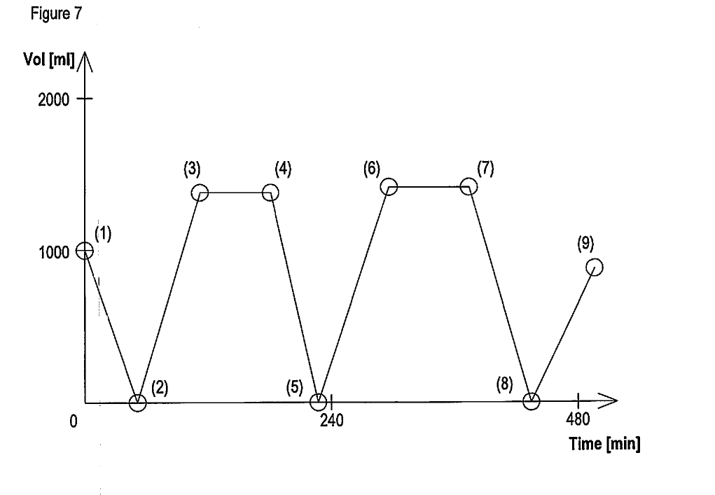

FIG. 7: a view in accordance with FIG. 2 having a darkened section of the treatment step already carried out.

The embodiment relates to a graphic editor in accordance with the invention in the form of a touch screen of a peritoneal dialysis machine.

The invention is, however, not restricted to peritoneal dialysis machines, but rather relates to any desired blood treatment devices and preferably to blood treatment devices having an extracorporeal blood circuit such as hemodialysis devices, hemodiafiltration devices and hemofiltration devices.

FIG. 1 shows a prescription progression drawn manually on the touch screen by a user of the device, wherein the volume of the dialysis solution which is present in the patient or is administered and removed from him is shown on the ordinate and the time is shown on the abscissa. This drawn prescription progression is processed into a prescription by a processor and is stored in a memory. This stored progression or a progression based thereon is then used as the basis for the peritoneal dialysis treatment.

A value such as the volume indicated at the point in time t=0 can be output and fine-tuned manually by touching the points or lines in order to exactly set the desired value, e.g. 1000 ml.

The manual input of values, however, takes place after the drawing of the progression by the user and not beforehand, as in the prior art, and the carrying out of the treatment or the preparation of the prescription in particular takes place without a prescription model having been stored beforehand, i.e. before the manual preparation of the prescription.

FIG. 2 shows the vectorization of the progression in accordance with FIG. 1, wherein the vectorization results in a linearization of the individual phases, in the present case the inflow phase, the dwell phase and the drain phase.

The user thus has the possibility of deleting the screen view in accordance with FIG. 1 and to vectorize the drawing so that the progression in accordance with FIG. 2 results.

Nine points are defined in this example, wherein the region between the points 1 and 2 represents the initial outflow, the section between the points 2 and 3 represents the inflow 1, the section between the points 3 and 4 represents the dwelling 1, the section between the points 4 and 5 represents the outflow 1, the section between the points 5 and 6 represents the inflow 2, the section between the points 6 and 7 represents the dwelling 2, the section between the points 7 and 8 represents the outflow 2 and the section between the points 8 and 9 represents the last inflow.

The prescription model can generally be further expanded by adding on further cycles.

The treatment can be changed at the request of the user with the help of these points, of the lines connecting the points and of touch gestures. If, for example, point 1 is dragged up or down by a finger or by a touch pen, etc., the initial outflow increases or reduces.

If the points 1 and 2 are touched and point 2 is dragged to the left or to the right, the duration for the initial outflow increases or decreases.

If the points 1 and 9 are touched and point 9 is dragged to the left or to the right, the total treatment duration increases or decreases accordingly. The complete treatment is uniformly horizontally stretched in so doing.

If point 1 and point 9 are touched and if point 1 and point 9 are dragged up or down, the treatment is stretched vertically. This means the initial outflow volume and also the total treatment volume, including the last inflow volume, change. The stretch ratio depends on the path length from point 1 to point 2.

If the user wants to leave the initial outflow volume unchanged and only wants to change the total treatment volume, including the last inflow volume, he touches the points 3 and 9 and drags the point 3 and the point 9 up or down. This has the consequence of a vertical stretching of the treatment. Only the total treatment volume, including the last inflow volume, changes. The stretch ratio depends on the path length from point 1 to point 2.

If the user wants to leave the initial outflow volume and the last inflow volume unchanged and only wants to change the volume for the inflows 1 and 2, he touches the points 3 and 7 and drags the points 3 and 7 up or down. This likewise has the consequence of a vertical stretching of the treatment. Only the total treatment volume changes. The stretch ratio depends on the path length from point 1 to point 2.

If the user only wants to change the volume of the first inflow, for example, he touches point 3 and drags it up or down. This has the consequence of a corresponding increase or reduction of the inflow volume.

The same applies accordingly to the increase or reduction of the second inflow volume by the touching and moving up or down of the point 6.

It is also possible to change the dwell times, i.e. the sections between the points 3 and 4 or 6 and 7. For this purpose, the user can, for example, touch the points 4 and 7 and drag point 7 to the left or to the right. This results in a uniform change of the dwell times 1 and 2.

It is also possible only to change one dwell time. For this purpose, the user e.g. only touches point 4 and drags it to the left or to the right, which has the consequence of a corresponding change of the dwell time 1. The same applies accordingly to the change of the dwell time 2. For this purpose, the user only touches point 7 and drags it to the left or to the right.

The type of change can generally depend on the number of the points touched on the touch screen, as can be seen by way of example from the above-named example.

The input prescription can also be changed with respect to the type of treatment.

The field “P” in FIG. 2 is a selection region which can be actuated by the user and its actuation tells the device that a disconnection phase is present in which the patient is not connected to the device.

FIG. 2a) shows a prescription progression having such a disconnection break, wherein the break lies between the points 3 and 4 in the example shown here, which is represented by a dashed line. The patient is not connected to the device in this time interval. A connection of the patient to the device is thus present up to point 3 and from point 4 onward. The further treatment cycles in FIG. 2a) are not provided with reference numerals for reasons of simplification and represent an exemplary progression of the treatment.

The field “i” in FIG. 2 is an information button which can be actuated by the user and on whose actuation information is output such as a prompt to act. This can, for example, relate to a user having to take medication. The information can also relate to the treatment itself; for example, the patient can be prompted to remove one or more dialyzate samples from the abdomen, for example to determine the peritoneal function. This determination can have an influence on the prescription to be administered to the patient.

Whereas FIG. 2 shows a standard peritoneal dialysis treatment, FIG. 3 shows a tidal cycle. This is achieved in that the dialysis volume is not completely removed from the patient, but only in part. The change takes place by the user in that he pushes the treatment point 5 up so that the progression in accordance with FIG. 3 results. Conversely, a switch can again be made to two standard cycles by dragging point 5 down in accordance with FIG. 3 as soon as the finger moves over the time axis.

It is also conceivable to change the type of the dispensed dialysis solution. For this purpose, the line between the points 2 and 3 can be touched, for example. In accordance with FIG. 4, the type of the solution (“solution 1”), its glucose content (here 4.25%) and the calcium ion content (here 1.75 mmol/l) is now displayed by this touch. If a parameter for the solution is to be changed, the button, e.g. for the solution, for the glucose concentration or also for the calcium concentration, can be selected. A selection list is displayed by the selection.

The value can be changed by selecting the list parameter so that the view shown in FIG. 5 results, for example. The user here has now selected solution 2 which has a glucose content of 2.3% and which has a calcium ion content of 1.25 mmol/l. After this selection, the corresponding section of the progression, in this case the inflow region between the points 2 and 3, can be changed by a color coding with respect to the original prescription.

It is also possible in accordance with the invention to change the number of cycles. If the user, for example, wants to remove the first cycle—as shown in FIG. 6—i.e. the region between the points 2 and 5, the user selects the points 2 and 5, i.e. touches them.

The selection fields “Add cycle” and “Remove cycle” then appear. If the user presses the field “Add cycle”, the cycle 1, i.e. the section between the points 2, 3, 4 and 5, is copied so that the prescription now has three cycles. If, on the other hand, the “Remove cycle” button is pressed, the cycle 1 is removed, i.e. the region between the points 2, 3, 4 and 5, so that the prescription subsequently only has one cycle.

FIG. 7 shows a screen view of the touch screen in which the already elapsed part of the treatment has a darker background than the still upcoming section.

In the embodiment shown here, a change of the treatment is only possible from point 5 onward. The treatment between the points 1, 2, 3 and 4 has already been completed so that a change by the user is no longer possible here. A touching of the touch screen thus has no consequences in this region. In the example in accordance with claim 7 shown here, thus only the last cycle and the last inflow volume can be changed, i.e. the section between the points 5, 6, 7 and 8, on the one hand, and between the points 8 and 9, on the other hand.

1. A blood treatment device having at least one touch screen for inputting data by the user of the blood treatment device and having at least one memory for storing a prescription, characterized in that the blood treatment device has at least one processor which is connected both to the touch screen and to the memory and which is configured to determine a prescription based on a progression drawn on the touch screen by the user, wherein the processor is furthermore configured only to determine this prescription based on the progression drawn by the user and without using values of the prescription stored in the memory in advance.

2. A blood treatment device in accordance with claim 1, characterized in that the progression is the temporal progression of one or more treatment parameters, wherein provision is preferably made that the treatment parameter is the volume of the dialysis solution and preferably the volume of the peritoneal dialysis solution present in the patient, one or more concentrations, the ultrafiltration rate or the dialysis dosage kT/V, the clearance, the filling volume and/or drain volume and/or the dwell time of the dialysis solution in the patient.

3. A blood treatment device in accordance with claim 1, characterized in that the processor is configured to carry out a vectorization of the drawn progression.

4. A blood treatment device in accordance with claim 1, characterized in that the processor is configured to determine a change to the prescription based on a change of the progression carried out on the touch screen by the user, with provision preferably being made that the change to the prescription relates to the change of the duration of the treatment, of the point in time of individual steps of the treatment, of the number of individual steps of the treatment, of the administered volume of a dialysis solution, of the drain volume or of the type of treatment.

5. A blood treatment device in accordance with claim 1, characterized in that the processor is configured to generate an output for adding or canceling treatment cycles and/or a output of one or more parameter values of the touched section on the touch screen based on a touching of a section of the progression carried out on the touch screen by the user, with provision preferably being made that the processor is configured such that it allows a change of the output parameter values on the touch screen.

6. A blood treatment device in accordance with claim 1, characterized in that the processor is configured such that it does not allow any changes to the prescription of the already carried out part of the treatment.

7. A method of preparing a prescription at a blood treatment device, wherein the blood treatment device has at least one touch screen for inputting data by the user of the blood treatment device and has at least one memory for storing the prescription, characterized in that the prescription is prepared in that the user draws the progression of the prescription on the touch screen; and in that the prescription is prepared without additionally making use of values of the prescription stored beforehand.

8. A method in accordance with claim 7, characterized in that the progression is the temporal progression of one or more treatment parameters, wherein provision is preferably made that the treatment parameter is the volume of the administered dialysis solution, preferably the volume of the peritoneal dialysis solution present in the patient, one or more concentrations, the ultrafiltration rate or the dialysis dosage kT/V, the clearance, the filling volume and/or drain volume and/or the dwell time of the dialysis solution in the patient.

9. A method in accordance with claim 7, characterized in that a vectorization of the drawn progression is carried out.

10. A method in accordance with claim 7, characterized in that a change to the prescription is determined based on a change of the progression carried out on the touch screen by the user, with provision preferably being made that the change to the prescription relates to the change of the duration of the treatment, of the point in time of individual steps of the treatment, of the number of individual steps of the treatment, of the administered volume of a dialysis solution or of the type of treatment.

11. A method in accordance with claim 7, characterized in that an output for adding or canceling treatment cycles and/or an output of one or more parameter values of the touched section on the touch screen takes/take place based on a touching of section of the progression carried out on the touch screen by the user, with provision preferably being made that a change to the output parameter values can be made on the touch screen by the user.

12. A method in accordance with claim 7, characterized in that no changes to the prescription of the already carried out part of the treatment are possible.
